(19) United States
(12) Patent Application Publication
(10) Pub. No.: US 2025/0336489 A1
(43) Pub. Date: Oct. 30, 2025

(54) MEDICAL TESTING APPARATUS AND MEDICAL TESTING METHOD

(71) Applicant: KONICA MINOLTA, INC., Tokyo (JP)

(72) Inventor: Takashi KIMOTO, Yokohama-shi (JP)

(21) Appl. No.: 19/088,941

(22) Filed: Mar. 24, 2025

(30) Foreign Application Priority Data
Apr. 25, 2024 (JP) 2024-071010

(51) Int. Cl. G16H 10/40; A61B 8/00; G16H 10/60

(52) U.S. Cl. CPC G16H 10/40; A61B 8/5223; G16H 10/60

(57) ABSTRACT

A medical testing apparatus includes: a communication unit communicably connected to a worklist server via a network; one or more processors configured to execute functions of: a testing information obtainer to obtain testing information (having patient information and a testing ID), which is used to order testing, from the worklist server; a display controller to cause a display operation unit to display the obtained testing information; a testing information editor to edit the obtained testing information; and a volatile storage unit to store the edited testing information edited by the testing information editor. The medical testing apparatus records the edited testing information temporarily stored in the volatile storage unit in a non-volatile storage unit, as edited testing information.

### CROSS-REFERENCE TO RELATED APPLICATION

This application claims the benefit of priority to Japanese Patent Application No. 2024-071010 filed on 25 Apr. 2024, the disclosures of all of which are hereby incorporated by reference in their entireties.

### TECHNICAL FIELD

The present invention relates to a medical testing apparatus and a medical testing method, particularly to a medical image testing apparatus, such as an ultrasonic diagnostic apparatus and an X-ray image diagnostic apparatus.

### DESCRIPTION OF RELATED ART

A medical image testing apparatus, such as an ultrasonic diagnostic device and an X-ray image diagnostic device, executes Modality Worklist Management (MWM). The modality worklist management is based on a specification of DICOM (Digital Imaging and Communications in Medicine) to manage operation of a testing apparatus (modality) making an inquiry to a worklist server, before testing, to obtain a worklist.

Japanese Patent Application Publication No. 2012-143380 A1 (hereinbelow, referred to as Patent Document 1) discloses an ultrasonic diagnostic device to obtain and register information from a server in parallel with ultrasonic testing. Additionally, Patent Document 1 describes that, when pieces of testing order information have been searched, the pieces of testing order information are popped up around an ultrasonic image, and a user selects specific testing order information from among the pieces of testing order information.

### SUMMARY OF THE INVENTION

When a testing apparatus receives an order for testing from a server, the order may have some fields in testing information having patient information (such as sex and name) left blank. If patient information is insufficient when browsed at the time of testing or after the testing, this may pose a risk of patients being mixed up or the like. Accordingly, it is desired to improve the reliability of testing information. In addition, in the case of an order from the modality worklist management, the worklist server holds the master information when testing is executed. In this case, a medical worker operating the testing apparatus, which has received the order, is not permitted to edit the patient information. Therefore, even if the patient information is partly left blank, the medical worker at the testing apparatus, which has received the order, cannot edit the patient information. The medical worker cannot complement missing patient information and testing information managed by the server.

The present invention has been devised in view of the above-described problem and is intended to provide a medical testing apparatus and a medical testing method capable of improving reliability of testing information.

To achieve at least one of the above-mentioned objective, a medical testing apparatus according to an aspect of the present invention includes: a communication unit communicably connected to a server via a network; one or more processors configured to execute functions of: a testing information obtainer to obtain testing information (having patient information and a testing ID), which is used to order testing, from the server; a display controller to cause a display unit to display the obtained testing information; and a testing information editor to edit the obtained testing information; and a storage unit to store (temporarily store) the edited testing information edited by the testing information editor.

### BRIEF DESCRIPTION OF DRAWINGS

The advantages and features provided by one or more embodiments of the invention can be fully understood from the detailed description given hereinbelow and the appended drawings which are given by way of illustration only and thus are not intended to define the limits of the present invention, in which: FIG. 1 shows a configuration of an image inspection system according to a first embodiment of the present invention;

FIG. 2 shows an example patient registration screen;

FIG. 3 is a flowchart of editing patient information;

FIG. 4 shows an example screen when editing patient information is started;

FIG. 5 shows an example screen used to edit patient information;

FIG. 6 is a flowchart of searching for edited patient information;

FIG. 7 is a flowchart of searching for patient information of the same patient, according to the first embodiment of the present invention; and

FIG. 8 is a flowchart of searching for patient information of the same patient, according to a second embodiment of the present invention.

### DETAILED DESCRIPTION

Hereinafter, one or more embodiments of the present invention are described with reference to the drawings. However, the scope of the invention is not limited to the disclosed embodiments. It should be noted that the drawings are merely schematic to the extent that the present invention can be fully understood. Accordingly, the present invention is not limited to those illustrated in the drawings. Additionally, common components and similar components in the drawings are denoted by the same reference signs, and duplicated descriptions thereof are skipped.

A medical testing apparatus 100 according to each of the embodiments of the present invention includes: a communication unit 50 communicably connected to a server (worklist server 200) via a network; a testing information obtainer 3 to obtain testing information 11 (having patient information and testing ID), which is used to order testing, from the server; a display controller 8 to cause a display unit (display operation unit 40) to display the obtained testing information 11; a testing information editor 4 to edit the obtained testing information 11; and a volatile storage unit 20 to store (temporarily store) edited testing information 12 edited by the testing information editor 4

### First Embodiment

FIG. 1 shows a configuration of an image inspection system according to a first embodiment of the present invention. In an image inspection system S, a medical testing apparatus 100 is communicably connected via a network with the worklist server 200 serving as a server, and an electronic medical record system 250 serving as a server. The medical testing apparatus 100 is an ultrasonic diagnostic device having an ultrasonic probe 1.

The worklist server 200 stores testing information having patient information in a non-volatile storage unit (not shown) and transmits the stored testing information to the medical testing apparatus 100. Here, the patient information is personal information such as a subject ID, as well as the name, sex, age, date of birth, height, and weight of the subject. The testing information includes a testing name, a testing ID, and the like, in addition to the patient information. In the present embodiment, a testing (ultrasonic diagnosis) is ordered through transmission of testing information (specifically a testing ID).

The electronic medical record system 250 is a medical information system unifying paper-based medical records and various testing results as electronic data. The electronic medical record system 250 may order testing (for example, image diagnosis) to the medical testing apparatus 100 in place of the worklist server 200.

The medical testing apparatus 100 is configured to include the ultrasonic probe 1, a control unit 10, the volatile storage unit 20 as a storage unit, a non-volatile storage unit 30 as a storage unit, the display operation unit 40 as a display unit, and a communication unit 50. The ultrasonic probe 1 is a sensor in which a plurality of ultrasonic transducers to generate ultrasonic waves and a plurality of wave receivers are arranged in a matrix.

The volatile storage unit 20 is a random access memory (RAM), and a storage area is secured therein to store the testing information 11 and edited testing information 12. The non-volatile storage unit 30 is a hard disk drive (HDD), a solid state disk (SSD), or the like, and a storage area is secured therein to store edited testing information 22. The display operation unit 40 is a liquid crystal panel (display unit) to allow for input operation via a touch system. The communication unit 50 is communicably connected to one or both of the worklist server 200 and electronic medical record system 250 via a local area network (LAN) or the like.

The control unit 10 is a central processing unit (CPU), and executes a program to implement functions of an image processor 2, the testing information obtainer 3, the testing information editor 4, a searcher 5, an editing determiner 6, a selector 7, and the display controller 8. The image processor 2 processes ultrasonic image signals outputted from the ultrasonic probe 1 and causes the display operation unit 40 to sequentially display testing images (ultrasonic diagnostic images). The testing information obtainer 3 obtains testing information having patient information from one or both of the worklist server 200 and electronic medical record system 250 via the communication unit 50. At this time, testing (ultrasonic diagnosis) is ordered. In addition, the testing information obtainer 3 stores the obtained testing information in the volatile storage unit 20 as the testing information 11.

The testing information editor 4 causes the display operation unit 40 to display and allow for editing the testing information obtained by the testing information obtainer 3 and the edited testing information 22 stored in the non-volatile storage unit 30. The testing information editor 4 of the present embodiment specifically allows for editing patient information included in the testing information. In addition, the testing information editor 4 stores the edited testing information in the volatile storage unit 20 as the edited testing information 12.

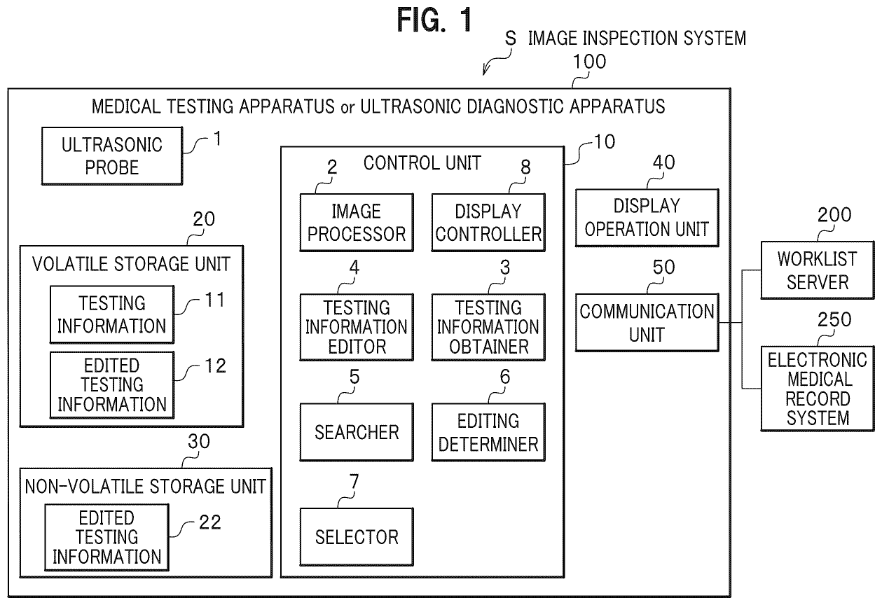

The searcher 5 searches the testing information 11 or edited testing information 12, stored in the volatile storage unit 20, for any field of the testing information (having the patient information). In particular, the searcher 5 of the present embodiment also functions as a same-patient searcher to search for the same patient. The editing determiner 6 determines whether or not the search result searched by the searcher 5 includes the edited testing information 12. That is, the editing determiner 6 determines whether the testing information (patient information) has been edited or not. The selector 7 selects one search result from among the plurality of search results searched by the searcher 5. The selector 7 causes the display operation unit 40 to display a plurality of search results, with a screen to allow a worker to select one. The display controller 8 causes the display operation unit 40 to display the screen (e.g., a patient registration screen 300 (see FIG. 2)) generated by the image processor 2, testing information editor 4, searcher 5, editing determiner 6, and selector 7. In addition, the display controller 8 outputs operation information operated by the display operation unit 40 to the above-described components.

FIG. 2 shows an example patient registration screen. The patient registration screen 300 is provided, at an upper left side thereof, with a testing “ID” field 301, “NAME” field 312, “DATE OF BIRTH” field 313, and “AGE” field 314. Here, the “NAME” field 312 is composed of a name-in-Kana field 302, name-in-Kanji field 303, and name-in-Alphabet field 304. In addition, the patient registration screen 300 is provided, in an upper middle thereof, with a “SEX” field 315, “TESTER” field 316, “HEIGHT” field 317, and “WEIGHT” field 318. The patient registration screen 300 is provided, at an upper right side thereof, with a current date and time field 320, “CLEAR” button 321, “EDIT” button 322, “START TESTING” button 323, and “CLOSE” button 324.

The patient registration screen 300 is provided, at a lower portion thereof, with the “TESTING DATE” field 345, “UPDATE” button 346, and table of “TESTING RESERVATION LIST.” The testing reservation list includes, in rows, a testing “ID” field 331, “NAME” field 332, “DATE OF BIRTH” field 333, “AGE” field 334, “SEX” field 335, “TESTING START DATE” field 336. The “TESTING RESERVATION LIST” is a list in which the “TESTING DATE” field 345 is set to “today” and lists in rows reserved testing for patients having the same testing start date of “2022 May 30.” Note that the “NAME” fields 332 are also identical.

Note that “PATIENT INFORMATION” in FIG. 2 is the testing information 11 obtained from one or both of the worklist server 200 and electronic medical record system 250 and stored in the volatile storage unit 20 (see FIG. 1). The worklist in the present embodiment is assumed to have only the name of “One Secondary Caputu” in alphabet or Roman letters and the sex of “other.” Accordingly, the “PATIENT INFORMATION” in the upper row has the name-in-Kana field 302 and name-in-Kanji field 303 left blank. In addition, the “SEX” field 315 in the upper row and the “SEX” field 335 of the “TESTING RESERVATION LIST” in the lower row each have “other” and coincide with each other.

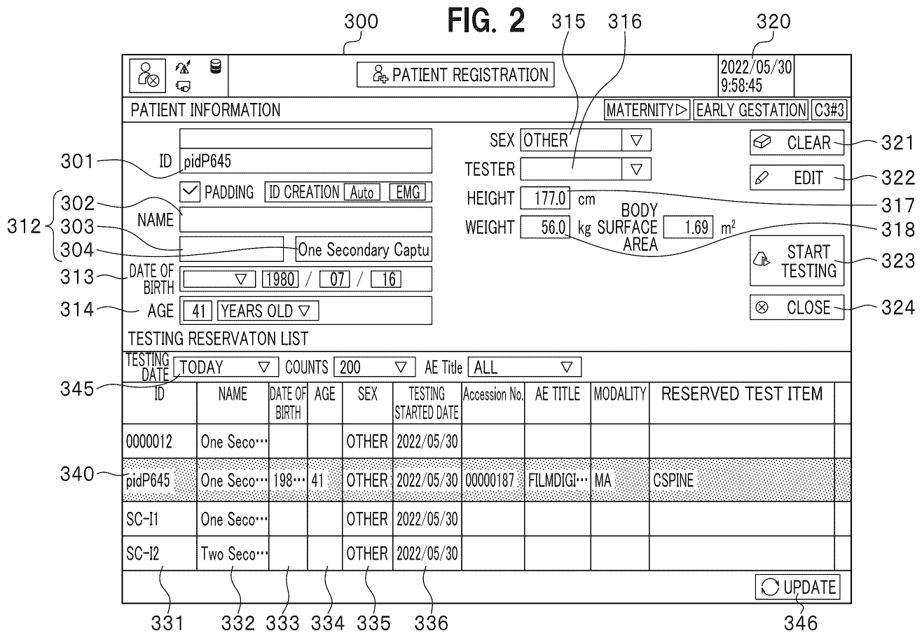

FIG. 3 is a flowchart of editing patient information. The worklist server 200 transmits testing information having patient information to the medical testing apparatus 100 (step S1). The communication unit 50 of the medical testing apparatus 100 receives the testing information having the patient information (step S2). At this time, the communication unit 50 of the medical testing apparatus 100 receives the testing ID to have the testing (ultrasonic diagnosis) ordered. Additionally, the testing information obtainer 3 (see FIG. 1) stores the received testing information in the volatile storage unit 20 as the testing information 11. After processing in step S2, the display controller 8 of the medical testing apparatus 100 causes the display operation unit 40 to display the testing information (having the patient information) received in step S2 (step S3). At this time, the display controller 8 of the medical testing apparatus 100 may cause the display operation unit 40 to display the edited testing information 22 stored in the non-volatile storage unit 30. For example, the display controller 8 of the medical testing apparatus 100 may cause the display operation unit 40 to display the above-described patient registration screen 300 (see FIG. 2).

After processing in step S3, a worker checks the patient registration screen 300 (see FIG. 2) and depresses the “EDIT” button 322 (step S4). The control unit 10 of the medical testing apparatus 100 determines whether or not the “EDIT” button 322 has been depressed (step S5). When the “EDIT” button 322 has not been depressed (“not depressed” in step S5), the control unit 10 of the medical testing apparatus 100 repeats the determination in step S5. When the “EDIT” button 322 has been depressed (“depressed” in step S5), the display controller 8 of the medical testing apparatus 100 causes the display operation unit 40 to display a confirmation message (step S6).

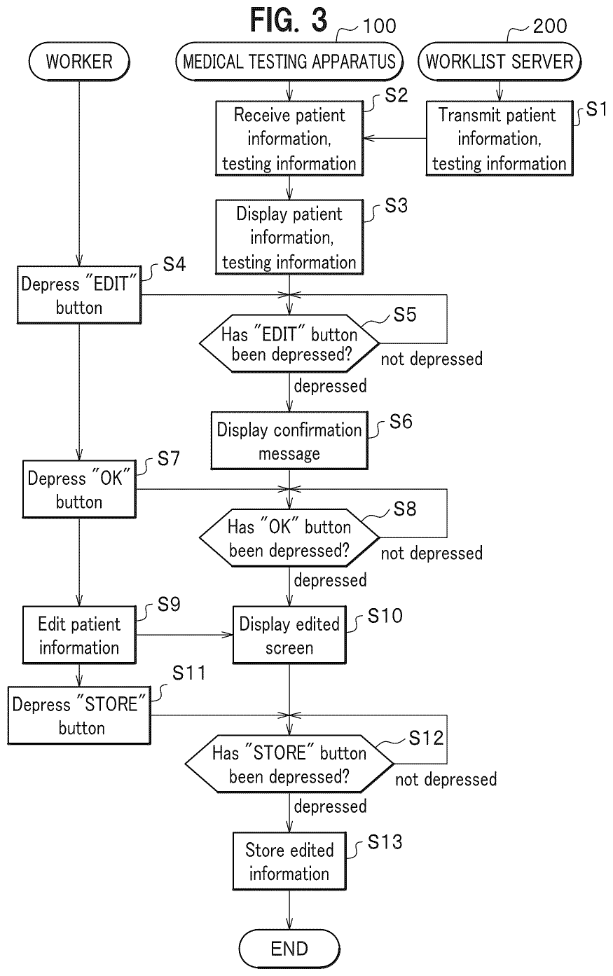

FIG. 4 shows an example screen when editing patient information is started. The screen in FIG. 4 is the patient registration screen 300 (see FIG. 2) having a message dialog box 350 popped up thereon. The message dialog box 350 includes character strings of “MESSAGE” and “Do you update PATIENT INFORMATION in TESTING RESERVATION LIST?”, an “OK” button 351, and a “CANCEL” button 352. Note that FIG. 4 illustrates a state of the “EDIT” button 322 having been depressed in step S4 (see FIG. 3).

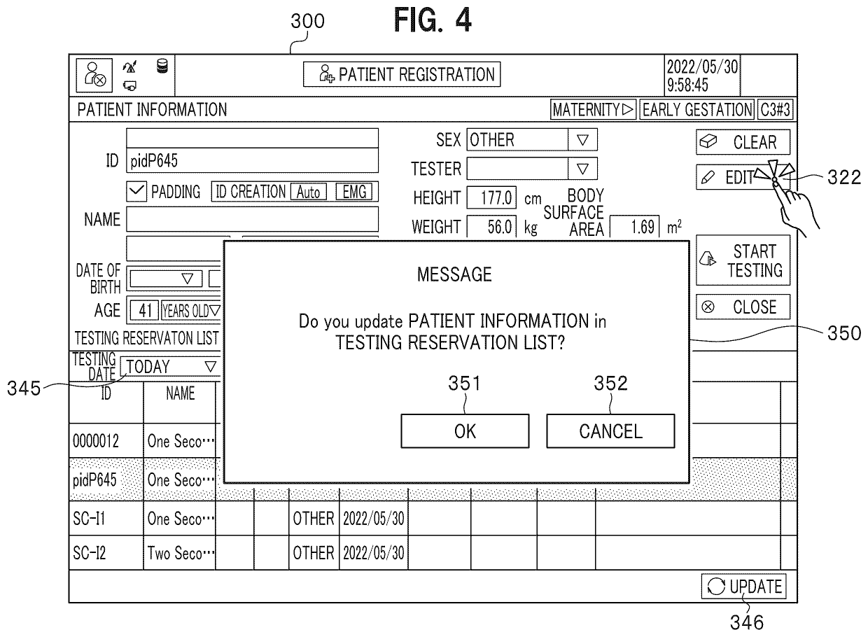

Returning to the flowchart in FIG. 3, the worker depresses the “OK” button 351 in the message dialog box 350 (step S7). After processing step S6, the control unit 10 of the medical testing apparatus 100 determines whether or not the “OK” button 351 has been depressed (step S8). When the “OK” button 351 has not been depressed (“not depressed” in step S8), the control unit 10 of the medical testing apparatus 100 repeats the determination in step S8. In contrast, when the “OK” button 351 has been depressed (“depressed” in step S8), the display controller 8 of the medical testing apparatus 100 causes the display operation unit 40 to display an edited screen 400 (see FIG. 5) (step S10).

FIG. 5 shows an example screen used to edit patient information. The edited screen 400 is configured similarly to the patient registration screen 300 (see FIG. 2). However, the edited screen 400 is provided with a “STORE” button 325 (see FIG. 5) and a “CANCEL” button 326 (see FIG. 5) in place of the “START TESTING” button 323 (see FIG. 2) and “CLOSE” button 324 (see FIG. 2). The “STORE” button 325 is a button to instruct whether or not to store the edited testing information 12 stored in the volatile storage unit 20, as the edited testing information 22 in the non-volatile storage unit 30. Accordingly, the edited screen (see FIG. 5) functions as a confirmation screen for confirming whether to record the edited testing information 12 in the non-volatile storage unit 30. That is, the display controller 8 causes the display operation unit 40 to display a confirmation screen to confirm whether or not to record the edited testing information in the non-volatile storage unit 30.

Returning to the flowchart in FIG. 3, the worker uses the edited screen 400 (see FIG. 5) to edit the patient information (step S9). For example, the worker may particularly add, at the time of medical interview, the name-in-Kana field 302 and the name-in-Kanji field 303 which have been left blank. Additionally, the worker may change the “SEX” field 315 from “OTHER” (FIG. 2) to “FEMALE.” This causes the testing information 11 (see FIG. 1) to be edited and causes the edited testing information 12 (see FIG. 1) to be stored in the volatile storage unit 20.

After editing the patient information (step S9), the worker depresses the “STORE” button 325 (step S11). After processing in step S10, the control unit 10 of the medical testing apparatus 100 determines whether or not the “STORE” button 325 has been depressed (step S12). When the “STORE” button 325 has not been depressed (“not depressed” in step S12), the medical testing apparatus 100 repeats the determination in step S12. In contrast, when the “STORE” button 325 has been depressed (“depressed” in step S12), the control unit 10 of the medical testing apparatus 100 stores (records) the edited patient information (edited testing information 12) stored in the volatile storage unit 20 (see FIG. 1) in the non-volatile storage unit 30 (FIG. 1), as the edited patient information (edited testing information 22) (step S13), and ends the process in FIG. 3.

Note that the edited testing information 22 may be transmitted (fed back) to one or both of the worklist server 200 and electronic medical record system 250. Here, whether or not the worklist being updated depends on the specification of the worklist server 200.

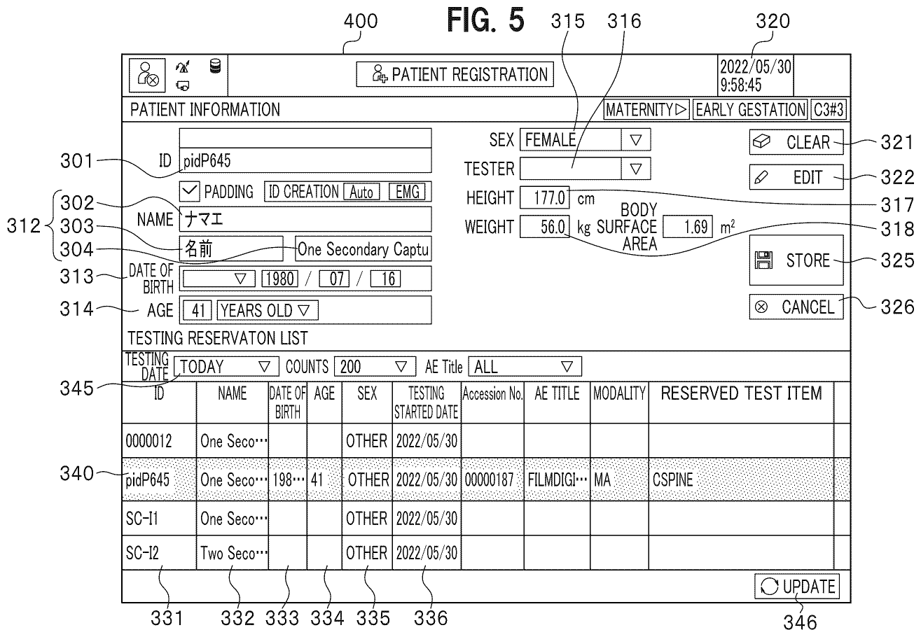

FIG. 6 is a flowchart of searching for edited patient information. This flow is activated by depressing a “SEARCH” button (not shown) or the like. The present embodiment is described with searching the “TESTING RESERVATION LIST” as an example. That is, the flow in FIG. 6 is activated when the worker sets “TODAY” in the “TESTING DATE” field 345 (see FIG. 2) and depresses the “UPDATE” button 346.

The searcher 5 of the medical testing apparatus 100 searches the edited testing information 12 stored in the volatile storage unit 20 (step S21) and causes the display operation unit 40 to display search results (step S22). For example, the “TESTING RESERVATION LIST” in FIG. 2 has the search results with the “TESTING START DATE” field 336 of “2022 May 30,” which is today, displayed in rows. The worker selects the edited patient information (step S23). For example, the worker selects a row 340 (see FIG. 2). The control unit 10 of the medical testing apparatus 100 determines whether or not any one has been selected from among a plurality of search results (step S24). When none has been selected (“No” in step S24), the control unit 10 of the medical testing apparatus 100 repeats the determination in step S24. In contrast, when one has been selected (“Yes” in step S24), the display controller 8 of the medical testing apparatus 100 causes the display operation unit 40 to display a screen (not shown) indicating the selected result (step S25), and the process in FIG. 6 ends.

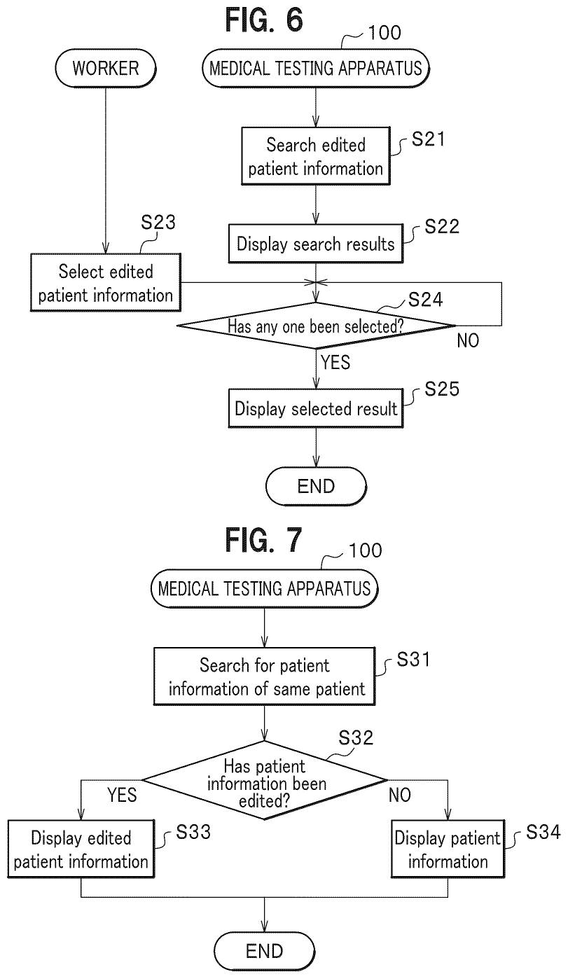

FIG. 7 is a flowchart of searching for patient information of the same patient, according to the first embodiment of the present invention. This flow is activated when a patient name is set and a “SAME PATIENT” button (not shown) is depressed. The searcher 5 of the medical test device 100 uses the patient information (testing information 11) stored in the volatile storage unit 20 to search for patient information (testing information) of the same patient as the subject (step S31). After processing in step S31, the control unit 10 of the medical testing apparatus 100 uses the edited patient information (edited testing information 12) to determine whether or not the patient information of the same patient has been edited (step S32). When the patient information has been edited (“Yes” in step S32), the display controller 8 of the medical testing apparatus 100 causes the display operation unit 40 to display the edited patient information (edited testing information 12) (step S33). In contrast, when the patient information has not been edited (“No” in step S32), the display controller 8 of the medical testing apparatus 100 causes the display operation unit 40 to display the patient information (testing information 11) (step S34), and the process in FIG. 7 ends.

As described above, the display controller 8 of the medical testing apparatus 100 according to the present embodiment causes the display operation unit 40 to display the testing information (having the patient information and the testing ID) obtained from one or both of the worklist server 200 and electronic medical record system 250. The medical testing apparatus 100 has testing ordered in response to receiving the testing information. When the “EDIT” button 322 (see FIG. 2) has been depressed, the medical testing apparatus 100 pops up the message dialog box 350 (see FIG. 4). When the “OK” button 351 (see FIG. 4) in the message dialog box 350 has been depressed, the displayed testing information 11 becomes editable.

The edited testing information 12 after editing is recorded in the non-volatile storage unit 30, as the edited testing information 22, when the “STORE” button 325 has been depressed. When the patient name in the worklist is only in alphabetic or Roman letters, the name-in-Kana field 302 and/or name-in-Kanji field 303 (see FIGS. 2 and 5) can be added through editing as well as recording in the non-volatile storage unit 30. This reduces the risk of patients being mixed up in subsequent testing with the medical testing apparatus 100.

The edited testing information 22 recorded in the non-volatile storage unit 30 is transmitted (fed back) by the communication unit 50 to one or both of the worklist server 200 and electronic medical record system 250. Here, whether or not the worklist being updated depends on the specification of the worklist server 200.

### Second Embodiment

In FIG. 7 of the first embodiment, the display controller 8 of the medical testing apparatus 100 causes the display operation unit 40 to display only one of the patient information (testing information 11) and the edited patient information (edited testing information 12) depending on whether or not the patient information has been edited. The display controller 8 may be configured to cause the display operation unit 40 to display the patient information (testing information 11) and edited patient information (edited testing information 12) and allow the worker to select one of the two.

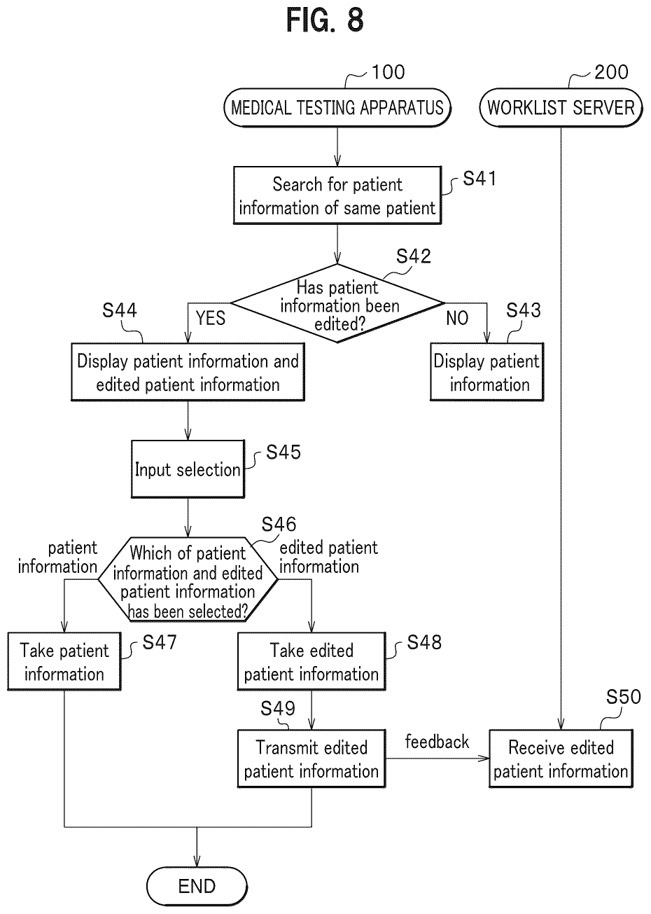

FIG. 8 is a flowchart of searching for patient information of the same patient, executed by a testing apparatus according to a second embodiment of the present invention. The searcher 5 of the medical test device 100 searches the patient information (testing information 11) stored in the volatile storage unit 20 for patient information of the same patient (step S41). After processing step S41, the editing determiner 6 of the medical testing apparatus 100 uses the edited patient information (edited testing information 12) to determine whether or not the patient information has been edited (step S42). When the patient information has not been edited (“No” in step S42), the display controller 8 of the medical testing apparatus 100 causes the display operation unit 40 to display the testing information 11 (step S43). In contrast, when the patient information has been edited (“Yes” in step S42), the display controller 8 of the medical testing apparatus 100 causes the display operation unit 40 to display both the testing information 11 and edited testing information 12 (step S44).

After processing in step 44, the selector 7 of the medical testing apparatus 100 receives a selection input of selecting whether to take the testing information 11 or to take the edited testing information 12 (step S45). After processing in step 45, the selector 7 of the medical testing apparatus 100 uses the selection input in step S45 to determine which of the testing information 11 and edited testing information 12 has been selected (step S46). When the testing information 11 has been selected (“Testing information” in step S46), the selector 7 of the medical testing apparatus 100 takes the testing information 11 (step S47), and the process ends. In contrast, when the edited testing information 12 has been selected (“Edited testing information” in step S46), the selector 7 of the medical testing apparatus 100 takes the edited testing information 12 (step S48). After processing in step 48, the communication unit 50 of the medical testing apparatus 100 transmits the edited testing information 12 to the worklist server 200 (step S49), and the process ends. The worklist server 200 receives the edited testing information 12 (step S50) for feedback. At this time, the worklist server 200 may or may not update the worklist.

### Modifications

The present invention is not limited to the above-described embodiments, and various modifications may be made within the scope of the present invention, as described below in case a).
a) The medical testing apparatus 100 of each of the embodiments described above is an ultrasonic diagnostic apparatus but may be an X-ray image diagnostic apparatus.

The present invention improves reliability of testing information.

Although the embodiments of the present invention have been described and illustrated in detail, the disclosed embodiments are for the purpose of illustration only and not for limitation. The scope of the present invention should be interpreted by terms of the appended claims.

### DESCRIPTION OF REFERENCE SIGNS

1: ultrasonic probe, 2: image processor, 3: testing information obtainer, 4: testing information editor, 5: searcher (same-patient searcher), 6: editing determiner, 7: selector, 8: display controller, 10: control unit, 11: testing information, 12: edited testing information, 20: volatile storage unit (storage unit), 22: edited testing information, 30: non-volatile storage unit (storage unit), 40: display operation unit (display unit), 50: communication unit, 100: medical testing apparatus (ultrasonic diagnostic apparatus), 200: worklist server (server), 250: electronic medical record system (server), 300: patient registration screen, 301: testing “ID” field, 312: “NAME” field, 315: “SEX” field, 322: “EDIT” button, 325: “STORE” button (button in confirmation screen), 336: “TESTING START DATE” field, 340: selected row, 350: message dialog box, 351: “OK” button, 400: edited screen, and S: image inspection system.

What is claimed is:

1. A medical testing apparatus comprising:
a communication unit communicably connected to a server via a network;
one or more processors configured to execute functions of:
a testing information obtainer to obtain testing information, which is used to order testing, from the server;
a display controller to cause a display unit to display the obtained testing information; and
a testing information editor to edit the obtained testing information; and
a storage unit to store the edited testing information edited by the testing information editor.

2. The medical testing apparatus according to claim 1, wherein
the testing information includes patient information.

3. The medical testing apparatus according to claim 1, wherein
the storage unit is a volatile storage unit, and
the display controller causes the display unit to display a confirmation screen to confirm whether or not to record the edited testing information in a non-volatile storage unit.

4. The medical testing apparatus according to claim 1, wherein
the communication unit transmits the edited testing information to the server to feed back the edited testing information.

5. The medical testing apparatus according to claim 1, wherein
the one or more processors are further configured to execute functions of:
a searcher to search for edited testing information; and
a selector to select testing information in search results,
wherein the display controller causes the display unit to display the testing information selected by the selector.

6. The medical testing apparatus according to claim 1, wherein
the one or more processors are further configured to execute functions of:
a same-patient searcher to search pieces of testing information, which have been obtained from the server, for testing information of the same patient; and
an editing determiner to determine whether or not the stored patient information has been edited,
wherein the display controller causes the display unit to display the edited patient information when the editing determiner has determined that the patient information has been edited.

7. The medical testing apparatus according to claim 6, wherein
the display controller causes both the information obtained from the server and the stored information to be displayed, when the editing determiner has determined that the patient information has been edited, and
the one or more processors are further configured to execute a function of:
a selector to allow for selecting one of two pieces of the displayed information to take.

8. A medical testing method executed by a medical testing apparatus communicably connected to a server via a network, the method comprising;
a testing information obtaining step of obtaining testing information, which is used to order testing, from the server;
a displaying step of causing a display unit to display the obtained testing information;
a testing information editing step of editing the obtained testing information; and
a storing step of storing the edited testing information, which has been edited in the testing information editing step, in a storage unit.
